(19) United States
(12) Patent Application Publication
(10) Pub. No.: US 2009/0041854 A1
(43) Pub. Date: Feb. 12, 2009

(54) PARATHYROID AND THYMUS TRANSPLANTATION IN DIGEORGE SYNDROME SUBJECTS

(76) Inventor: M. Louise Markert, Durham,, NC (US)

Correspondence Address: MYERS BIGEL SIBLEY & SAJOVEC, PO BOX 37428, RALEIGH, NC, 27627, US

(21) Appl. No.: 11/816,125

(22) PCT Filed: Mar. 22, 2006
(86) PCT No.: PCT/US2006/010387

§ 371 Date: Apr. 24, 2008

Related U.S. Application Data
(60) Provisional application No. 60/668,745, filed on Apr. 6, 2005.
(60) Provisional application No. 60/709,534, filed on Aug. 19, 2005.

(51) Int. Cl. A61K 35/26; C12N 5/02; C12N 5/08; A61P 3/00

(52) U.S. Cl. 424/580; 435/383; 435/372

(57) ABSTRACT

A method of treating hypoparathyroidism in a human DiGeorge syndrome subject comprises (a) implanting thymus tissue into the subject in an amount effective to treat the DiGeorge syndrome; and (b) implanting, preferably concurrently implanting, parathyroid tissue into the subject in an amount effective to treat the hypoparathyroidism.

### FIELD OF THE INVENTION

The present invention concerns the treatment of hypoparathyroidism occurring in subjects afflicted with DiGeorge syndrome.

### BACKGROUND OF THE INVENTION

DiGeorge syndrome is a heterogeneous condition in which infants are born with variable deficiences of the thymus, parathyroid, and heart and often have other anomalies as well. In complete DiGeorge syndrome infants are athymic and are born with a profound T cell deficiency. DiGeorge syndrome can be treated by thymus transplantation, as described in M. Louise Markert et al., Thymus transplantation in complete DiGeorge syndrome: immunologic and safety evaluations in 12 subjects, Blood 102, 1121-1130 (2003) and M. Louise Markert et al., Transplantation of Thymus tissue in complete DiGeorge syndrome, N Eng. J. Med. 341, 1180-9 (1999).

Approximately 20% of complete DiGeorge syndrome infants have profound hypoparathyroidism. In addition, about ¾ of cases have hypoparathyroidism in general. Although thymus transplantation results in reconstitution of T cells, the ongoing hypoparathyroidism is a significant problem. The infant must take calcium replacement several times a day and is at risk of nephrocalcinosis and hypocalcemic seizures. It would be extremely useful to provide alternate ways of addressing the problem of hypoparathyroidism in DiGeorge subjects.

### SUMMARY OF THE INVENTION

A first aspect of the present invention is a method of treating hypoparathyroidism in a human DiGeorge syndrome subject. The method comprises (a) implanting thymus tissue into the subject in an amount effective to treat the DiGeorge syndrome; and (b) implanting, preferably concurrently implanting, parathyroid tissue into the subject in an amount effective to treat the hypoparathyroidism.

A second aspect of the invention is an organ culture medium, particularly a thymus culture medium, characterized in that the organ culture medium is free of 2-deoxyguanosine.

A third aspect of the invention is the use of an organ culture medium, particularly a thymus culture medium, as described above, for carrying out a method as described herein.

A further aspect of the invention is a method of preparing live human thymus tissue for implantation into a subject in need thereof, comprising: (a) culturing live human thymus tissue under sterile conditions in a culture medium free of 2-deoxyguanosine for a time period sufficient to deplete mature T cells therefrom (typically at least eight days), and then (b) collecting the live human thymus tissue from the culture medium for implantation into a subject in need thereof (e.g., a DiGeorge syndrome subject).

A further aspect of the invention is isolated and cultured live human thymus tissue that is both free of exogenous 2-deoxyguanosine and depleted of mature T cells (e.g., is prepared by a method as described above).

The foregoing and other objects and aspects of the present invention are explained in greater detail in the specification set forth below.

### BRIEF DESCRIPTION OF THE DRAWINGS

FIG. 1 shows parathyroid hormone (PTH) levels in picograms per milliliter for the patient prior to transplantation (at day 0) and through 120 days after transplantation.

FIG. 2 shows ionized calcium levels for the patient of FIG. 1 prior to, during and after transplantation, with all supplemental calcium being stopped at day 104.

FIG. 3 shows T cell function, assessed by PHA responses, for the patient of FIGS. 1-2, as compared to a normal adult control.

FIG. 4. Parathyroid hormone levels in patient DIG201 after thymus/parathyroid transplantation.

FIG. 5. Development of T cells in patient DIG201 after thymus parathyroid transplantation. T cells have developed with normal phenotype.

### DETAILED DESCRIPTION OF THE PREFERRED EMBODIMENTS

“Hypoparathyroidism” as described herein is a deficiency of parathyroid hormone that causes abnormal metabolism of calcium and phosphorus. Signs of hypoparathyroidism may include one or more of low serum calcium level, elevated serum phosphorus, decreased serum parathyroid hormone level, decreased serum magnesium level, and/or abnormal heart rhythms. Symptoms of hypoparathyroidism include one or more of tingling of lips hands and feet, muscle cramps, pain in face legs or feet, abdominal pain, dry hair, brittle nails, dry scaly skin, cataracts, delayed or absent tooth formation, weakened tooth enamel, hand or foot spasms, muscle spasms or tetany, and/or convulsions or seizures.

The term “treat” as used herein refers to any type of treatment that imparts a benefit to a subject afflicted with a disease, including improvement in the condition of the subject (e.g., in one or more symptoms, such as decrease the frequency and/or severity of hypocalcemic seizures), delay in the progression of the disease, etc.

“Concurrently implanting” as used herein means that the two tissues or organs are implanted at the same point in time or one immediately following the other. In the latter case, the two organs or tissues are administered at times sufficiently close that the results observed are essentially or substantially indistinguishable from those achieved when the organs or tissues are implanted at the same time.

“Depleted of mature T cells” as used herein means that the number of CD3+ T cells in the thymus has been decreased so that less than or equal to 10% of the starting number (the number found in the material to be cultured prior to culturing) remains.

1. Thymus preparation and implantation. Thymus tissue can be cultured and prepared for implantation in accordance with known techniques, such as described in L. Market et al., Blood 102, 1121-1130 (2003). Culturing of the tissue is carried out to deplete the thymus of mature T cells that may otherwise contribute to graft vs. host disease in the implanted subject, for example by including 2-deoxyguanosine in the culture medium. Id. In general such culture techniques are carried out by harvesting thymus in a sterile fashion, slicing the thymus tissue with a microtome into slices approximately 0.1 to 5 (most preferably 0.5) millimeters thick, placing the slices on a carrier or support medium or matrix (e.g., filters such as MILLIPORE™ filters on surgical sponges) and in turn placing the carrier or support in organ medium in a container such as a Petri dish, still under sterile conditions, to be cultured therein. The culture media generally comprises nutrients sufficient to sustain the viability of the tissue during culturing therein, at least one antibiotic sufficient to combat microbial infection of the tissue, and a buffer system sufficient to maintain an appropriate pH range for the tissue during culture. A preferred thymus organ medium comprises or consists essentially of F12 nutrient mixture (Ham; Gibco, Grand Island, N.Y.) with 1.36 mM 2-deoxyguanosine (Sigma, St. Louis, Mo.), 25 mM HEPES (N-2-hydroxyethylpiperazine-N′-2-ethanesulfonic acid; Gibco), 2 mM L-glutamine (Gibco), 10% fetal bovine serum (GIBCO), 100 ug/mL streptomycin sulfate (Gibco), 1 ug/mL gentamycin (Gibco) and 100 ug/dL Amphotericin B (BeniaSicor, Irvine, Calif.). The slices are maintained in a 5% CO₂ incubator at 37° C. The medium is preferably changed daily. The tissue is cultured for at least 8, 9, 10 or 11 days, up to 3 or 4 weeks or more.

In an alternate embodiment of the invention disclosed herein, thymus tissue is collected and cultured in essentially the same manner as described above, except that the 2-deoxyguanosine is eliminated from the culture medium through the entire culturing period. An advantage of this embodiment is there is no damage to the thymic tissue from the administration of 2-deoxyguanosine and there is no residual in the cultured thymus tissue that may be transferred to the recipient. While not wishing to be bound to any particular theory of this embodiment, it is believed that depletion of mature T cells from the thymus prior to implantation is achieved by the amount of time (at least eight or ten days, up to four weeks or more, as noted above) the thymus tissue is maintained in the culture medium.

2. Parathyroid preparation and implantation. Parathyroid tissue need not be cultured as described above and instead may be collected from a donor and implanted directly into a subject without intervening culturing or chemical processing steps, as described in J. Olson et al., Ann. Surg. 223, 472-487 (2002) and C. Lo, ANZ J. Surg. 72, 902-907 (2002). In one embodiment the parathyroid is minced or sliced prior to implantation.

3. Subjects and implantation. Subjects to be treated by the methods of the present invention are, in general, human subjects, including both male and female subjects. In general the subjects are not more than 2 years old, and typically the subjects are not more than 18 months old.

Subjects suitable for the treatments or methods of the invention include both complete DiGeorge syndrome subjects who are athymic who also have profound hypoparathyroidism and complete DiGeorge syndrome subjects who have partial parathyroid deficiency as evidenced by their requiring calcium or calcitriol supplementation to prevent hypocalcemia.

Some DiGeorge syndrome subjects exhibit T cell function that poses a risk of allotransplant rejection if no immunosupression is given. Hence the subjects are preferably treated or conditioned with an immunosupressive agent prior to implantation of thymus and parathyroid to reduce the risk of transplant rejection. While any suitable immunosupressive treatment can be used, a preferred treatment is to administer anti-thymocyte antibodies (e.g., rabbit anti-thymocyte globulin) to the subjects in a dose or dosages (e.g., three separate doses) prior to (e.g., at least one day before) the implantation, in an amount effective to combat transplant rejection of the thymus and parathyroid tissue, as described in L. Markert et al., Blood 104, 2574-2581 (2004).

Thymus tissue prepared as described herein is preferably implanted into a muscle of the subject, preferably a skeletal muscle such as quadriceps, in accordance with standard surgical procedures, preferably with the subject under local or general anesthesia. In general the amount of thymus tissue implanted is from 1 gram or 1.5 grams up to 6, 10 or 20 grams per subject (based upon the assumption that one cubic centimeter of tissue equals one gram). The thymus tissue is optionally but preferably matched to the subject for at least one of the three major HLA loci (HLA-A, HLA-B, and HLA-DR).

The parathyroid tissue is preferably implanted into a muscle of the subject, again preferably a skeletal muscle such as a quadriceps, in accordance with standard surgical procedures, preferably with the subject under local or general anesthesia. The parathyroid tissue is preferably implanted concurrently with, or during the same surgical session as, the thymus tissue (e.g., when general anesthesia is employed, during the same session of general anesthesia), though it need not be, and preferably is not, implanted in the same location as the thymus tissue. The parathyroid tissue is preferably matched to the subject for at least one of the three major HLA loci (HLA-A, HLA-B, and HLA-DR), and preferably is closely matched, e.g., is obtained from either a mother or father f the subject.

In general a single adult parathyroid gland is a sufficient quantity of parathyroid for implantation, although a smaller amount can be used when minced or sliced tissue is used (e.g., from 20 or 40%, by weight or volume, up to the entire quantity of the parathyroid gland).

Subjects may receive cyclosporine and/or steroids in the peri transplantation period, depending upon the patient's immune status, in accordance with known techniques. Pneumocystis prophylaxis and/or intravenous immunoglobulin may be used for one or two years post transplantation, also in accordance with known techniques.

While the present invention has been described with reference to the transplantation of parathyroid tissue, it will be appreciated that other solid organs, including but not limited to heart and lung, can also be transplanted concurrently with thymus tissue instead of parathyroid tissue as described herein, to thereby reduce or inhibit rejection of the transplanted organ in an infant with complete DiGeorge syndrome.

The present invention is explained in greater detail in the following non-limiting examples.

### EXAMPLE 1

### DiGeorge Syndrome Patient 1

A female infant was diagnosed with DiGeorge syndrome based on hypoparathyroidism associated with a hypocalcemic seizure on day 11, ventricular septal defects requiring surgery at 1 month, and absence of T cells noted at 5 weeks of age. The 22q11 FISH test was normal.

An unrelated postnatal thymus was obtained for transplantation. The thymus shared an HLA-DR allele with the mother and the infant. The mother was used as the parathyroid donor. Her HLA types along with those of the thymus donor and recipient are shown in the Table below.

| | HLA-A | HLA-B | HLA-C | HLA-DRB1 |
|---|---|---|---|---|
| Patient | 0201 | 3501 | 0401 | 0101 |
| | 0301 | 3508 | 0401 | 1401 |
| Thymus | 0101 | 3501 | 0401 | 0101 |
| | 1101 | 1801 | 0501 | 0301 |
| Parathyroid | 0201 | 3501 | 0401 | 0101 |
| | 0401 | 1801 | 0701 | 0701 |

Rabbit anti-human thymocyte globulin was used as pre-transplant conditioning. Thymus slices were inserted into the quadriceps of the recipient in an open procedure after a 2-week culture period as described in M. Markert et al., Blood 102, 1121-1130 (2003). Concurrent parathyroid transplantation was performed at the time of the thymus transplant. Donor parathyroid tissue was obtained from the mother in an open operative procedure. The parathyroid was minced into small pieces and inserted into the quadriceps at a site distinct from the thymus graft, as described in Lo et al., ANZ J. Surg. 72, 902-907 (2002). Parathyroid graft function was assessed by monitoring serum calcium and intact parathyroid hormone (PTH).

Results: There were no adverse events for the recipient or parathyroid donor relating to the transplants. The recipient has had normal PTH levels on all days post transplantation when tested. The initial test was on day 17 and the most recent test was on day 143. The patient is not on any calcium supplementation. The thymus graft biopsy at 2.5 months showed thymopoiesis (T cell development in the thymus graft). On day 157 the patient had 18% T cells with a PHA response of 205,326 counts per minute (cpm) with a background medium response of 142 cpm. This is a normal level T cell response. Remarkably on day 138, the patient had a central line infection without associated hypocalcemia or seizure. This line has been removed.

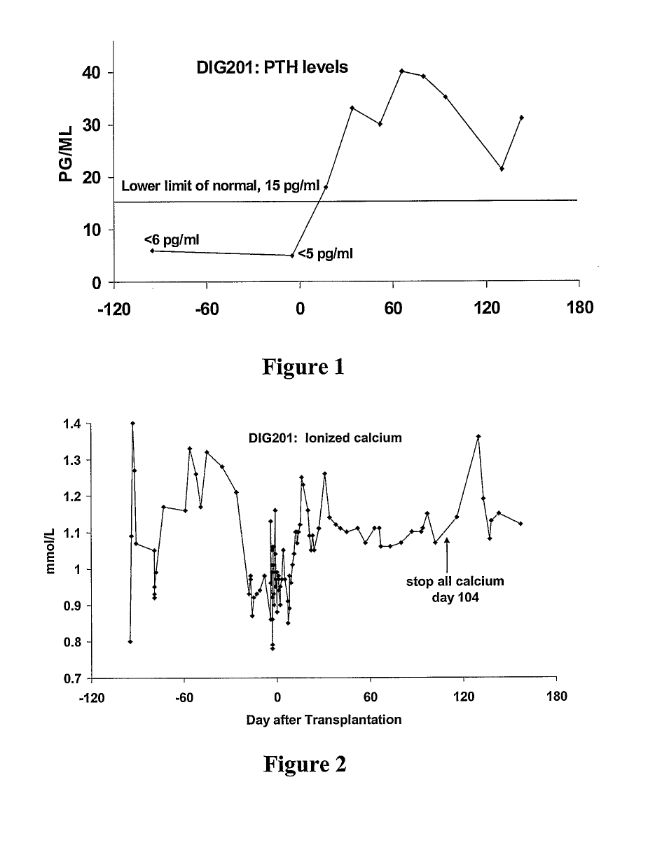

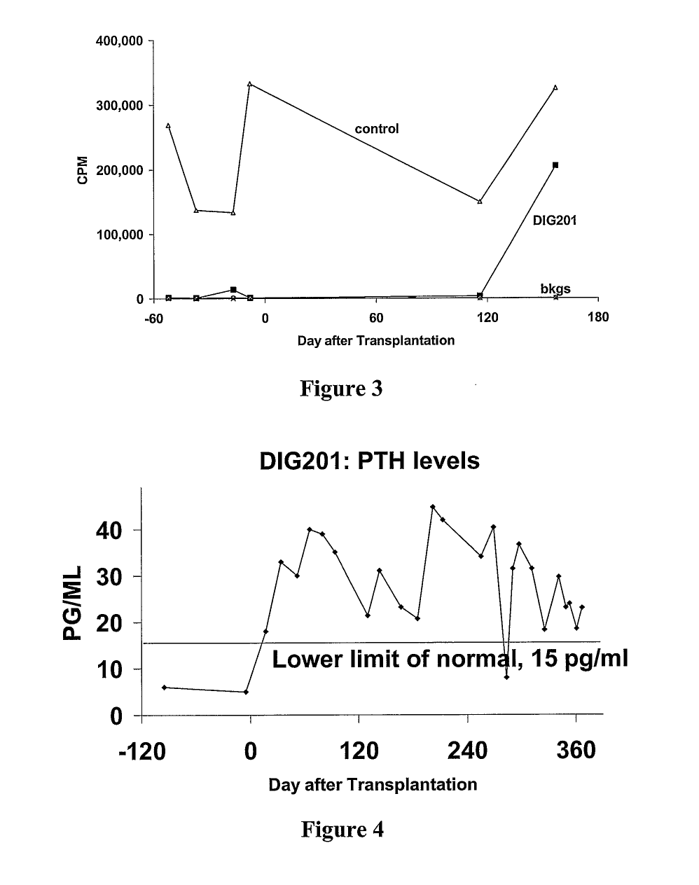

The graphs of FIGS. 1-3 show the PTH and T cell function data for the patient as discussed above.

### EXAMPLE 2

### Follow-Up on Patient 1 and Additional Patients

After Example 1 above, parathyroid and thymus transplantation was done in three additional infants for a total of 4 infants with complete DiGeorge syndrome (DIG201, DIG203, DIG204, and DIG206). As of this writing the patients have not had seizures from hypocalcemia after parathyroid transplantation, nor have they developed nephrocalcinosis.

There have been no adverse events associated with the parathyroid donation or the transplantation.

For patient DIG204, the parathyroid transplant was given approximately one month after thymus transplantation because the donor had a thyroid nodule that had to be biopsied prior to use of a parathyroid gland from that parent. Thus, the parental parathyroid donor had two operative procedures, one for the thyroid nodule biopsy and a second for the parathyroid organ donation. The recipient also had two operative procedures, one for thymus transplantation and one for parathyroid transplantation. The thymus was transplanted as soon as the tissue was available because T cell function is so important for the survival of the patient.

All infants have been able to come off calcium supplementation. Two patients (DIG201, DIG203) have required occasional supplementation.

DIG201 was put on calcium supplementation for approximately one week when her ionized calcium fell to 0.93 mmol/L. This has happened twice and was associated with diarrhea both times. However, without the parathyroid transplantation, it would have been necessary to keep her ionized calcium around 0.9 mmol/L to prevent nephrocalcinosis.

DIG203 was put on calcium supplementation when his calcium level fell to 7.9 mg/dl. He currently, however, has normal calcium levels without supplementation.

DIG204 has not been on calcium supplementation since her transplantation.

DIG206 was able to wean off calcium in the first two weeks after parathyroid transplantation.

Parathyroid hormone levels have increased in all patients (the level from Mar. 6, 2006 from the 4th patient who was transplanted Feb. 22, 2006 is pending). See FIG. 4 for sample parathyroid hormone levels in DIG201.

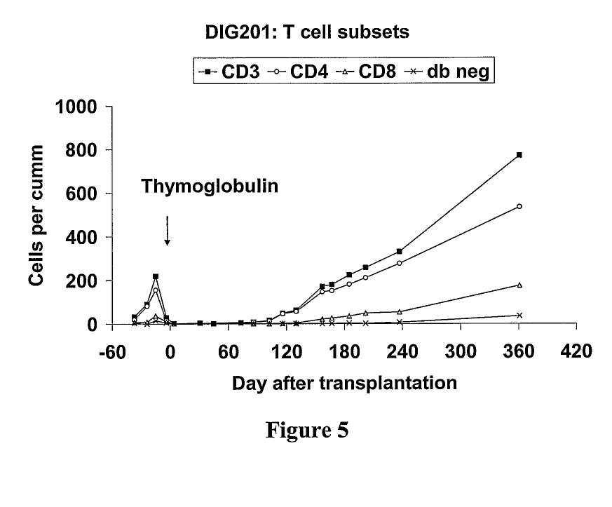

Thymic derived T cells have developed in the initial three patients (DIG201, DIG203, and DIG204). DIG206 is too close to transplantation to have developed T cells. This was a critical issue. It does not appear that the parathyroid transplant adversely affects the thymus transplant. See FIG. 5 for development of T cells in DIG201. Not shown is the normal proliferative responses of the T cells to mitogens.

Regarding the use of thymus organ culture medium that is free of 2-deoxyguanosine. The concern about use of culture medium free of 2-deoxyguanosine is that of graft-versus-host disease. We transplanted an infant (DIG034) using thymus cultured without 2-deoxyguanosine in the medium. The resulting thymus tissue was depleted of viable T cells and no GVHD has developed to date. The quality of the thymus tissue for transplantation is much improved without the 2-deoxyguanosine. In particular, the viability of the thymus tissue is improved in general and specifically the viability of thymic epithelium is improved.

The foregoing is illustrative of the present invention, and is not to be construed as limiting thereof. The invention is defined by the following claims, with equivalents of the claims to be included therein.

That which is claimed is:

1. A method of treating hypoparathyroidism in a human DiGeorge syndrome subject, comprising:
(a) implanting human thymus tissue into said subject in an amount effective to treat said DiGeorge syndrome; and concurrently
(b) implanting human parathyroid tissue into said subject in an amount effective to treat said hypoparathyroidism.

2. The method of claim 1, wherein said thymus tissue comprises 1 to 20 grams of thymus tissue.

3. The method of claim 1, wherein said parathyroid tissue comprises at least 20% of one adult parathyroid.

4. The method of claim 1, wherein said subject is a complete DiGeorge syndrome subject.

5. The method of claim 1, wherein said subject is a complete DiGeorge syndrome subject with partial parathyroid function and afflicted with hypoparathyroidism.

6. The method of claim 1, wherein said subject is not more than two years old.

7. The method of claim 1, further comprising the step of administering said subject anti-human thymocyte globulin prior to said step of (a) implanting human thymus tissue.

8. The method of claim 1, wherein said human thymus tissue is free of 2-deoxyguanosine.

9. The method of claim 1, wherein said implanting step (a) is carried out by implanting human thymus tissue into a skeletal muscle of said subject.

10. The method of claim 1, wherein said implanting step (b) is carried out by implanting human parathyroid tissue into a skeletal muscle of said subject.

11. A method of preparing live human thymus tissue for implantation into a subject in need thereof, comprising:
(a) culturing live human thymus tissue under sterile conditions in a culture medium free of 2-deoxyguanosine for a time period of at least eight days to thereby deplete mature T cells therefrom, and then
(b) collecting said live human thymus tissue from said culture medium for implantation into a subject in need thereof.

12. The method of claim 11, wherein said thymus tissue is sliced or minced.

13. The method of claim 11, wherein said culturing step is carried out for a time of 10 days to four weeks.

14. Isolated and cultured live human thymus tissue produced by the process of claim 10.

15. Isolated and cultured live human thymus tissue free of exogenous 2-deoxyguanosine and depleted of mature T cells.
